(19) United States
(12) Patent Application Publication
(10) Pub. No.: US 2025/0206638 A1
(43) Pub. Date: Jun. 26, 2025

(54) Chemical Control Systems and Methods for Detecting Abnormal Operation of Chloramine Residual Control

(71) Applicant: cleanwater1, inc., Milpitas, CA (US)

(72) Inventor: Robin Giguere, Piedmont, CA (US)

(21) Appl. No.: 18/988,663

(22) Filed: Dec. 19, 2024

Related U.S. Application Data
(60) Provisional application No. 63/613,276, filed on Dec. 21, 2023.

(51) Int. Cl. C02F 1/00; C02F 1/50; G01N 33/18

(52) U.S. Cl. CPC C02F 1/008; C02F 1/50; G01N 33/18; C02F 2209/003; C02F 2209/14; C02F 2209/29; C02F 2303/04

(57) ABSTRACT

A method of detecting abnormal operation includes: (a) determining residual chloramine concentration in a water sample obtained from the body of water; (b) determining an average rate of change in total chlorine concentration when the residual chloramine concentration is below a predetermined target chloramine concentration level; (c) automatically engaging a supply of ammonia and a supply of chlorine to add both ammonia and chlorine to the body of water at a weight ratio of chlorine to ammonia of 5:1 or less when the average rate of change in total chlorine concentration is below a first set rate of change in total chlorine concentration; and (d) initiating an alarm to indicate an abnormal operation of chloramine residual in the body of water if, after step c), an average rate of change in total chlorine concentration is below a second set rate of change in total chlorine concentration.

### CROSS-REFERENCE TO RELATED APPLICATIONS

This application claims the benefit of U.S. Provisional Patent Application No. 63/613,276, filed Dec. 21, 2023, the disclosure of which is incorporated herein by reference in its entirety.

### BACKGROUND OF THE INVENTION

### Field of the Invention

The present invention relates to chemical control systems for controlling disinfectants and detecting abnormal operation of chloramine residual, and methods of operating such systems and detecting the abnormal operation of chloramine residual control.

### Description of Related Art

Water utilities typically add disinfectants to water systems to prevent contamination from germs and bacteria. The most commonly used secondary disinfectant is chlorine; however, while chlorine is a strong disinfectant, it has a short residual life and readily forms disinfection byproducts such as trihalomethanes. In order to avoid the drawbacks associated with chlorine, many water utilities are turning to chloramines as an alternative. As compared to chlorine, chloramines have a longer residual life and are less prone to disinfection byproduct formation.

Despite these advantages, chloramine also naturally degrades as water ages in the distribution system due to a series of reversible chemical reactions. Degradation of chloramine additionally releases free ammonia during the degradation process that is used by nitrifying bacteria as a food source. Because of the undesirable side-effects from chloramine degradation, chemical injection systems have been developed to control and produce stable forms of monochloramine in a water system at a desired concentration.

Current chloramine control systems typically share the same process goals of increasing the chloramine residual over time and reaching a desired chlorine residual. As such, these systems help prevent degradation of the chloramine and the undesirable side-effects. While these control systems can prevent chloramine degradation, abnormal operation of the system could still result in chloramine degradation. Thus, it is desirable to provide a chemical injection system that can be accurately controlled to continuously produce stable forms of monochloramine in a water system at a desired concentration and which is also able to automatically detect abnormal operation of the system.

### SUMMARY OF THE INVENTION

In one preferred and non-limiting embodiment or aspect, provided is a method of automatically detecting abnormal operation of chloramine residual control in a body of water contained in a reservoir. The method includes: (a) determining residual chloramine concentration in a water sample obtained from the body of water; (b) determining an average rate of change in total chlorine concentration when the residual chloramine concentration is below a predetermined target chloramine concentration level; (c) automatically engaging a supply of ammonia and a supply of chlorine to add both ammonia and chlorine to the body of water at a weight ratio of chlorine to ammonia of 5:1 or less when the average rate of change in total chlorine concentration is below a first set rate of change in total chlorine concentration; and (d) initiating an alarm to indicate an abnormal operation of residual chloramine in the body of water if, after step c), an average rate of change in total chlorine concentration is below a second set rate of change in total chlorine concentration.

In certain preferred and non-limiting embodiments or aspects, the first set rate of change in total chlorine concentration is equal to or greater than the second set rate of change in total chlorine concentration. In some preferred and non-limiting embodiments or aspects, the average rate of change in total chlorine concentration can also be determined by measuring the change in residual total chlorine concentration in consecutively obtained water samples over a fixed period of time. The residual chloramine concentration can also be based on a residual total chlorine concentration.

In certain preferred and non-limiting embodiments or aspects, the average rate of change in total chlorine concentration in step d) is determined after a time-delay following step c). The alarm can also be initiated in step d) when the following algorithm is satisfied:

$${\frac{dTCL}{dt}}_{C} < {\frac{dTCL}{dt}}_{min} and elapsed > TD = initiate alarm ,$$

wherein

$${\frac{dTCL}{dt}}_{C}$$

is the observed average rate of change in total chlorine concentration,

$${\frac{dTCL}{dt}}_{min}$$

is the second set rate of change in total chlorine concentration, elapsed is the time elapsed before determining the average rate of change in total chlorine concentration that is compared to the second set rate of change in total chlorine concentration, and TD is the time delay for abnormal detection.

In certain preferred and non-limiting embodiments or aspects, the method further includes automatically engaging a supply of chlorine only to add only chlorine to the body of water, or engaging a supply of ammonia and a supply of chlorine to add both ammonia and chlorine to the body of water at a weight ratio of chlorine to ammonia of greater than 5:1, prior to step b) if the residual chloramine concentration in the water sample obtained from the body of water in step a) is below the predetermined target chloramine concentration level.

In one preferred and non-limiting embodiment or aspect, provided is a treatment delivery and control system for detecting abnormal operation of chloramine residual control in a body of water contained in a reservoir. The system includes: a chemical dosing assembly; a water sampling assembly configured to extract a water sample from the body of water at different points in time; one or more analyzers in fluid communication with the water sampling assembly and configured to determine at least total chlorine concentration in the water samples; a controller in operable communication with the one or more analyzers; and one or more computer-readable storage mediums in operable communication with the controller and containing programming instructions that, when executed, cause the controller to: (a) determine residual chloramine concentration in a water sample obtained from the body of water; (b) determine an average rate of change in total chlorine concentration when the residual chloramine concentration is below a predetermined target chloramine concentration level; (c) automatically engage a supply of ammonia and a supply of chlorine to add both ammonia and chlorine to the body of water at a weight ratio of chlorine to ammonia of 5:1 or less when an average rate of change in total chlorine concentration is below a first set rate of change in total chlorine concentration; and (d) initiate an alarm to indicate an abnormal operation of chloramine residual in the body of water if, after step b), an average rate of change in total chlorine concentration is below a second set rate of change in total chlorine concentration.

In certain preferred and non-limiting embodiments or aspects, the chemical dosing assembly is at least partially submerged in the body of water. In some preferred and non-limiting embodiments or aspects, the first set rate of change in total chlorine concentration is equal to or greater than the second set rate of change in total chlorine concentration. The average rate of change in total chlorine concentration can be determined by measuring the change in residual total chlorine concentration in consecutively obtained water samples over a fixed period of time. Further, the residual chloramine concentration can be based on a residual total chlorine concentration.

In some preferred and non-limiting embodiments or aspects, the average rate of change in total chlorine concentration in step d) is determined after a time-delay following step c). The alarm can be initiated in step d) when the following algorithm is satisfied:

$${\frac{dTCL}{dt}}_{C} < {\frac{dTCL}{dt}}_{min} and elapsed > TD = initiate alarm ,$$

wherein

$${\frac{dTCL}{dt}}_{C}$$

is the observed average rate of change in total chlorine concentration,

$${\frac{dTCL}{dt}}_{min}$$

is the second set rate of change in total chlorine concentration, elapsed is the time elapsed before determining the average rate of change in total chlorine concentration that is compared to the second set rate of change in total chlorine concentration, and TD is the time delay for abnormal detection.

In some preferred and non-limiting embodiments or aspects, the one or more computer-readable storage mediums in operable communication with the controller further contains programming instructions that, when executed, cause the controller to automatically engage a supply of chlorine only to add only chlorine to the body of water, or engage a supply of ammonia and a supply of chlorine to add both ammonia and chlorine to the body of water at a weight ratio of chlorine to ammonia of greater than 5:1, prior to step b) if the residual chloramine concentration in the water sample obtained from the body of water in step a) is below the predetermined target chloramine concentration level.

Additional preferred and non-limiting embodiments or aspects are set forth and described in the following clauses.

Clause 1: A method of automatically detecting abnormal operation of chloramine residual control in a body of water contained in a reservoir, the method comprising: (a) determining residual chloramine concentration in a water sample obtained from the body of water; (b) determining an average rate of change in total chlorine concentration when the residual chloramine concentration is below a predetermined target chloramine concentration level; (c) automatically engaging a supply of ammonia and a supply of chlorine to add both ammonia and chlorine to the body of water at a weight ratio of chlorine to ammonia of 5:1 or less when the average rate of change in total chlorine concentration is below a first set rate of change in total chlorine concentration; and (d) initiating an alarm to indicate an abnormal operation of chloramine residual in the body of water if, after step c), an average rate of change in total chlorine concentration is below a second set rate of change in total chlorine concentration.

Clause 2: The method of clause 1, wherein the first set rate of change in total chlorine concentration is equal to or greater than the second set rate of change in total chlorine concentration.

Clause 3: The method of clause 1 or 2, wherein the average rate of change in total chlorine concentration is determined by measuring the change in residual total chlorine concentration in consecutively obtained water samples over a fixed period of time.

Clause 4: The method of any one of clauses 1-3, wherein the residual chloramine concentration is based on a residual total chlorine concentration.

Clause 5: The method of any one clauses 1-4, wherein the average rate of change in total chlorine concentration in step d) is determined after a time-delay following step c).

Clause 6: The method of clause 5, wherein the alarm is initiated in step d) when the following algorithm is satisfied:

$${\frac{dTCL}{dt}}_{C} < {\frac{dTCL}{dt}}_{min} and elapsed > TD = initiate alarm ,$$

wherein

$${\frac{dTCL}{dt}}_{C}$$

is the observed average rate of change in total chlorine concentration,

$${\frac{dTCL}{dt}}_{min}$$

is the second set rate of change in total chlorine concentration, elapsed is the time elapsed before determining the average rate of change in total chlorine concentration that is compared to the second set rate of change in total chlorine concentration, and TD is the time delay for abnormal detection.

Clause 7: The method of any one of clauses 1-6, further comprising automatically engaging a supply of chlorine only to add chlorine to the body of water, or engaging a supply of ammonia and a supply of chlorine to add both ammonia and chlorine to the body of water at a weight ratio of chlorine to ammonia of greater than 5:1, prior to step b) if the residual chloramine concentration in the water sample obtained from the body of water in step a) is below the predetermined target chloramine concentration level.

Clause 8: A treatment delivery and control system for detecting abnormal operation of chloramine residual control in a body of water contained in a reservoir comprising: a chemical dosing assembly; a water sampling assembly configured to extract water sample from the body of water at different points in time; one or more analyzers in fluid communication with the water sampling assembly and configured to determine at least total chlorine concentration in the water samples; a controller in operable communication with the one or more analyzers; and one or more computer-readable storage mediums in operable communication with the controller and containing programming instructions that, when executed, cause the controller to: (a) determine residual chloramine concentration in a water sample obtained from the body of water; (b) determine an average rate of change in total chlorine concentration when the residual chloramine concentration is below a predetermined target chloramine concentration level; (c) automatically engage a supply of ammonia and a supply of chlorine to add both ammonia and chlorine to the body of water at a weight ratio of chlorine to ammonia of 5:1 or less when an average rate of change in total chlorine concentration is below a first set rate of change in total chlorine concentration; and (d) initiate an alarm to indicate an abnormal operation of chloramine residual in the body of water if, after step b), an average rate of change in total chlorine concentration is below a second set rate of change in total chlorine concentration.

Clause 9: The system of clause 8, wherein the chemical dosing assembly is at least partially submerged in the body of water.

Clause 10: The system of clauses 8 or 9, wherein the first set rate of change in total chlorine concentration is greater than or equal to the second set rate of change in total chlorine concentration.

Clause 11: The system of any one of clauses 8-10, wherein the average rate of change in total chlorine concentration is determined by measuring the change in residual total chlorine concentration in consecutively obtained water samples over a fixed period of time.

Clause 12: The system of any one of clauses 8-11, wherein the residual chloramine concentration is based on a residual total chlorine concentration.

Clause 13: The system of any one of clauses 8-12, wherein the average rate of change in total chlorine concentration in step d) is determined after a time-delay following step c).

Clause 14: The system of clause 13, wherein the alarm is initiated in step d) when the following algorithm is satisfied:

$${\frac{dTCL}{dt}}_{C} < {\frac{dTCL}{dt}}_{min} and elapsed > TD = initiate alarm ,$$

wherein

$${\frac{dTCL}{dt}}_{C}$$

is the observed average rate of change in total chlorine concentration,

$${\frac{dTCL}{dt}}_{min}$$

is the second set rate of change in total chlorine concentration, elapsed is the time elapsed before determining the average rate of change in total chlorine concentration that is compared to the second set rate of change in total chlorine concentration, and TD is the time delay for abnormal detection.

Clause 15: The system of any one of clauses 8-14, wherein the one or more computer-readable storage mediums in operable communication with the controller further contains programming instructions that, when executed, cause the controller to automatically engage a supply of chlorine only to add chlorine to the body of water, or engage a supply of ammonia and a supply of chlorine to add both ammonia and chlorine to the body of water at a weight ratio of chlorine to ammonia of greater than 5:1, prior to step b) if the residual chloramine concentration in the water sample obtained from the body of water in step a) is below the predetermined target chloramine concentration level.

### BRIEF DESCRIPTION OF THE DRAWINGS

FIG. 1 illustrates a treatment delivery system according to the principles of the present invention;

FIG. 2 illustrates a chemical dosing assembly according to the principles of the present invention;

FIG. 3 is a chloramine breakpoint curve;

FIG. 4 depicts graphs illustrating the addition of chlorine in the presence of free ammonia to generate chloramine; and

FIG. 5 depicts graphs illustrating the addition of chlorine and ammonia in the absence of free ammonia to generate chloramine.

### DETAILED DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Further, the terms “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the specification, are simply exemplary embodiments of the invention. Hence, specific dimensions and other physical characteristics related to the embodiments disclosed herein are not to be considered as limiting.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances.

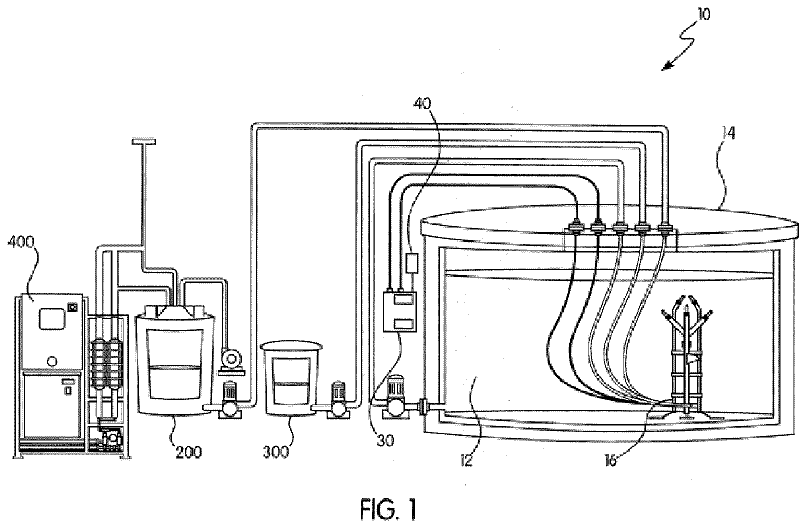

Referring to FIG. 1, in one preferred and non-limiting embodiment or aspect, the present invention is directed to a treatment delivery system 10 that can be used to automatically control chloramine concentration in a body of water 12 contained in a reservoir 14. The term “automatic control” refers to the absence of substantial participation of a human operator in normal operations manually controlling the controllable components. As such, the treatment delivery system 10 can be controlled without an operator monitoring or adjusting the various parameters of the treatment delivery system 10 during normal operations.

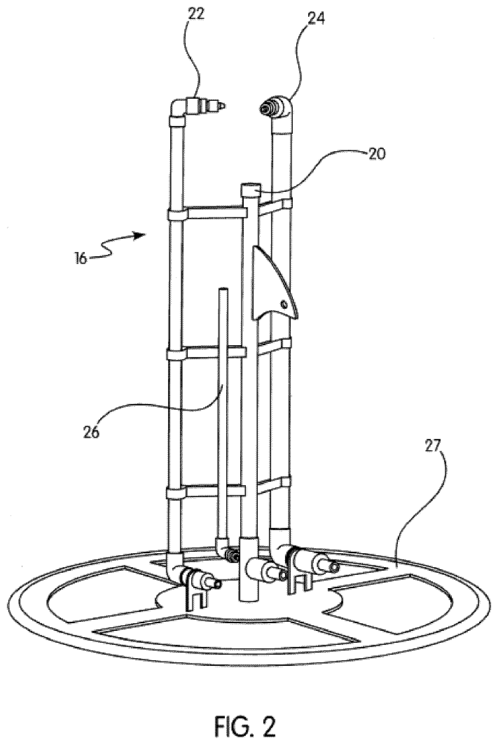

As shown in FIG. 1, the treatment delivery system 10 can include a chemical dosing assembly 16 that can be at least partially submerged in the body of water 12. Referring to FIG. 2, and in one preferred and non-limiting embodiment or aspect, the chemical dosing assembly 16 can include a water motive tube 20, a first chemical treatment flow tube 22, and a second chemical treatment flow tube 24. The water motive tube 20 and chemical treatment flow tubes 22, 24 of the chemical dosing assembly 16 can be oriented to expel water and chemicals, respectively, into the body of water 12 held in the reservoir 14. The chemicals used with the chemical treatment tubes 22, 24 can be selected to form chloramine, such as monochloramine, when expelled into a jet of water expelled from the water motive tube 20. For example, the first chemical treatment flow tube 22 can be in fluid communication with a source of chlorine and can be configured to expel chlorine into the body of water 12 while the second chemical treatment flow tube 24 can be in fluid communication with a source of ammonia and can be configured to expel ammonia into the body of water 12. Because of the configuration of the nozzle ends of the first and second chemical treatment flow tubes 22, 24, the chemicals expelled through the ends thereof come into almost immediate contact with one another and can begin reacting soon after being expelled into the body of water 12.

In addition, in one preferred and non-limiting embodiment or aspect, the water motive tube 20 is positioned below the release point of the first and second chemical treatment flow tubes 22, 24 to circulate the chemicals into the body of water 12. The flow of water out of the water motive tube 20 can also create a high energy, high velocity mixing zone directly above the water motive tube 20 where the chemicals can be released, which helps the chemicals interact and form a particular compound, such as monochloramine. The treatment delivery system 10 can include multiple chemical dosing assemblies 16 strategically located throughout the reservoir 14.

The treatment delivery system 10 can further include a water sampling assembly 26 that is configured to obtain or extract water samples from the body of water 12 at different points in time, such as continuously, periodically, and/or according to a pre-programmed cycle. As shown in FIG. 2, the water sampling line 26 can be a component of the chemical dosing assembly 16. For example, the water motive tube 20, chemical treatment tubes 22, 24, and water sampling assembly 26 of the chemical dosing assembly 16 can be secured to a frame 27 that is adapted to rest at the bottom of the reservoir 14. Alternatively, the water motive tube 20, the chemical treatment tubes 22, 24, and the water sampling assembly 26 can extend into the reservoir 14 to a desired depth. Yet another alternative (not shown) is that the water sampling assembly 26 can be separate from the chemical dosing assembly 16 and may be located at any location within the reservoir 14. Treatment delivery system 10 may also include multiple water sampling assemblies 26 positioned throughout the reservoir 14. It is appreciated that the water sample can also be obtained from other methods including, but not limited to, water sampling with a submersible pump positioned inside the reservoir 14.

Referring to FIG. 1, and in one preferred and non-limiting embodiment or aspect, the treatment delivery system 10 can also include one or more analyzers 30 that are in fluid communication with the water sampling assembly 26. The analyzer(s) 30 are configured to receive the water samples and analyze the contents thereof in order to determine the chloramine concentration. Various methods are known to determine the chloramine concentration in a sample of water. In one preferred and non-limiting embodiment or aspect, the analyzer(s) 30 are programmed or configured to determine the concentration of total chlorine of the water sample.

The present invention can also include analyzer(s) 30 programmed to determine other parameters including, but not limited to, pH, temperature, and combinations thereof. It will be appreciated that the analyzer(s) 30 may be a standalone device or, in other embodiments, may be software and/or firmware executed by the controller 40 or other processor.

In one preferred and non-limiting embodiment or aspect, the analyzer 30 is, or includes, a total chlorine analyzer, such as the total chlorine analyzer commercially available from ProMinent Fluid Controls, Inc. of Pittsburgh, PA, which can be used to indirectly measure the chloramine concentration. The analyzer(s) 30 can also be configured to measure other chemicals and components in the water such as, for example, oxidation-reduction potential which reflects the ability of certain chemical components in the water to accept or lose electrons. It is appreciated that the total chlorine residual in a water sample is used to determine the residual chloramine concentration either by the analyzer 30 or by a controller 40 or other processor associated therewith. In some preferred and non-limiting embodiment or aspect, the analyzer(s) 30 is, or also includes, a chloramine analyzer, such as the APA 6000 Ammonia and Monochloramine Analyzer commercially available from Hach Company of Loveland, CO, which can directly measure the chloramine concentration in the water sample.

As indicated, the treatment delivery system 10 can further include a controller 40 that is in operable communication with the analyzer(s) 30 so that measurements and other data gathered, and/or determined by the analyzer(s) 30, can be transferred to or accessed by the controller 40. One or more computer-readable storage mediums can be in operable communication with the controller 40. The computer-readable storage mediums can contain programming instructions that, when executed, cause the controller 40 to perform multiple tasks. This includes programming algorithms such as those described herein that allow the controller 40 to control the administration of chlorine and/or ammonia into the body of water 12 for establishing, reestablishing, and maintaining target residual chloramine levels within the body of water 12. The programming instructions can be updated and modified. For example, the target residual chloramine level can be changed as can the flow rates of the chlorine and/or ammonia and the water sampling frequency.

In one example, and in one preferred and non-limiting embodiment or aspect, the programming instructions, when executed, can cause the controller 40 to: measure and/or analyze a water sample obtained from the body of water 12, and/or determine whether the residual chloramine concentration in the water sample is below a predetermined residual chloramine concentration set-point or below a chloramine concentration percentage of a predetermined residual chloramine concentration set-point (both which are also referred to herein as a “predetermined chloramine concentration target level”); when the residual chloramine concentration is below a predetermined chloramine concentration target level, determine an average rate of change in total chlorine concentration based on residual total chlorine concentrations of water samples obtained from the body of water; automatically engage a supply of ammonia and a supply of chlorine to add both ammonia and chlorine to the body of water at a weight ratio of chlorine to ammonia of 5:1 or less when the average rate of change in total chlorine concentration is below a first set rate of change in total chlorine concentration; and initiate an alarm to indicate an abnormal operation of residual chloramine in the body of water if an average rate of change in total chlorine concentration is below a second set rate of change in total chlorine concentration. It is appreciated that controller 40 may include one or more microprocessors, CPUs, and/or other computing devices.

As further shown in FIG. 1, and in one preferred and non-limiting embodiment or aspect, treatment delivery system 10 can include multiple chemical storage tanks, such as a first chemical storage tank 200 and a second chemical storage tank 300, which are configured to transport chemicals to the chemical dosing assembly 16 via one or more metering pumps. As indicated, the treatment delivery system 10 can deliver a source of chlorine and a source of ammonia into the body of water 12. As such, the chemical storage tanks 200, 300 can store a source of chlorine and a source of ammonia. Because the treatment delivery system 10 is capable of delivering any type of chlorine and ammonia source, the chemical storage tanks 200, 300 can be selected to store various sources of chlorine and ammonia. Non-limiting examples of chlorine sources that can be used with the present invention include pressurized chlorine gas and hypochlorites such as sodium hypochlorite, potassium hypochlorite, and calcium hypochlorite. Non-limiting examples of ammonia sources that can be used with the present invention include pressurized anhydrous ammonia, aqueous ammonia, and liquid ammonium sulfate. The chemical storage tanks 200, 300 can also be supplied by on-site chemical generation systems, such as an on-site hypochlorite generation system 400 as shown in FIG. 1 for example that can generate hypochlorite based chemicals (e.g., sodium hypochlorite or potassium hypochlorite) directly at the water treatment site.

Non-limiting examples of chemical dosing assemblies, chemical generation systems, and the like are disclosed in U.S. Pat. No. 9,039,902, which is incorporated by reference herein in its entirety. In particular, U.S. Pat. No. 9,039,902 describes chemical dosing assemblies, as well as a hypochlorite generation system, that can be used as the source of chlorine that is present in first chemical storage tank 200 and, ultimately, supplied to the body of water 12. The treatment delivery system 10 can also utilize other mixing systems as well. For example, the treatment delivery system 10 can also utilize the mixing system disclosed in U.S. Pat. No. 7,862,302, which is incorporated by reference herein in its entirety.

As indicated, the present disclosure is also directed to a method of automatically detecting abnormal operation of chloramine residual control in a body of water 12 contained in a reservoir 14. As used herein, “abnormal operation of chloramine residual control” refers to the situation when chloramine residual decreases in response to chemical dosing of a body of water. For instance, abnormal operation of chloramine residual control can occur when chloramine residual continues to decrease after adding chlorine and ammonia at a weight ratio of chlorine to ammonia of 5:1 or less. The method can be implemented through one or more algorithms and controls contained in programming instructions that, when executed, cause the system 10 to take certain actions, as described below.

The method can include measuring, analyzing, and/or determining the residual chloramine concentration in a water sample obtained from the body of water 12. The water sample can be obtained with the water sampling assembly 26 and transported to the analyzer 30 that is in fluid communication with the water sampling assembly 26. The analyzer 30 can then measure, analyze, and/or determine the residual chloramine concentration. The determination of the residual chloramine concentration can include measuring the residual total chlorine concentration in the water sample.

The residual chloramine concentration determination can be reported to a controller 40 that is in operable communication with one or more computer-readable storage mediums. The controller 40 also has knowledge of, or access to, information about the predetermined chloramine concentration target level, which can be based on a residual total chlorine concentration target level, for example. In some preferred and non-limiting embodiments or aspects, if the residual chloramine concentration in the water sample is determined to be below the predetermined chloramine concentration target level (which can be based on a residual total chlorine concentration set-point, for example), additional water samples are obtained from the body of water 12 to determine the average rate of change in total chlorine concentration based on residual total chlorine concentrations of the water samples.

As used herein, the “average rate of change in the total chlorine concentration” refers to the change in the total chlorine concentration value over time based on the total chlorine concentration in two or more water samples.

In some preferred and non-limiting embodiments or aspects, the average rate of change in total chlorine concentration is determined by comparing the total chlorine concentration in a plurality of water samples obtained after a fixed period of time. For example, the average rate of change in total chlorine concentration can be based on the average change in total chlorine concentration between consecutively obtained water samples over a specified period of time, such as 10 minutes or 30 minutes or one hour for example.

The rate of change in the total chlorine concentration is determined by the least squares regression. Non-limiting examples of such equations are described in Slope Filtering: An FIR Approach to Linear Regression, IEEE SIGNAL PROCESSING MAGAZINE, November 2008, pages 159 to 163. The rate of change in the total chlorine concentration can also be determined by the following formula: average rate of change=last determined total chlorine concentration first determined total chlorine concentration value/time the water sample of the last determined value was obtained−time the water sample of the first determined value was obtained. Thus, the programming instructions can include the average rate of change formula to allow the controller 40 to determine the average rate of change in total chlorine concentration in the body of water 12.

After determining the average rate of change in total chlorine concentration, ammonia and chlorine are both added to the body of water 12 at a weight ratio of chlorine to ammonia of 5:1 or less if the average rate of change in total chlorine concentration is below a first set rate of change in chloramine concentration. The chlorine and ammonia can be added to the body of water 12 as previously described through treatment tubes 22 and 24 of the chemical dosing assembly 16.

As used herein, a “set rate of change in total chlorine concentration” refers to a predetermined (target) increase or decrease in the rate of change in total chlorine concentration. For example, the set rate of change in total chlorine concentration can be 0.05 mg/L/hour and if the average rate of change in residual total chlorine concentration is determined to be below 0.05 mg/L/hour, the programming instructions will cause the controller 40 to automatically engage (or control) a supply of chlorine and ammonia to add both ammonia and chlorine to the body of water 12 at a weight ratio of chlorine to ammonia of 5:1 or less.

It is appreciated that the set rate of change in total chlorine concentration can be based on a positive or negative rate of change. As used herein, a “positive rate of change” refers to an increase in the total chlorine concentration over a period of time, and a “negative rate of change” refers to a decrease in the total chlorine concentration over a period of time.

In some preferred and non-limiting embodiments or aspects, the method step of adding both chlorine and ammonia into the body of water 12 at a weight ratio of chlorine to ammonia of 5:1 or less is controlled by the following algorithm: (1) w<y=add both chlorine and ammonia at a weight ratio of chlorine to ammonia of 5:1 or less, where “w” is the rate of change in total chlorine concentration determined from the water samples and “y” is the set rate of change in total chlorine concentration. Thus, the programming instructions can include the previous algorithm that, when satisfied, will cause the controller 40 to automatically engage (or control) a supply of chlorine and a supply of ammonia to add both chlorine and ammonia to the body of water 12 at a weight ratio of chlorine to ammonia of 5:1 or less.

After adding ammonia and chlorine to the body of water 12 at a weight ratio of chlorine to ammonia of 5:1 or less, an alarm will be initiated if a later measured average rate of change in total chlorine concentration is below a second set rate of change in total chlorine concentration when the supply of chlorine and ammonia are disengaged, or while chlorine and ammonia are added to the body of water 12. The second set rate of change in total chlorine concentration refers to a second predetermined (target) increase or decrease in the rate of change in total chlorine concentration. The second set rate of change in total chlorine concentration can be different than the first set rate of change in total chlorine concentration. For example, the first set rate of change in total chlorine concentration can be equal to or greater than the second set rate of change in total chlorine concentration. In such examples, if the average rate of change in total chlorine concentration continues to decrease and drops below the second set rate of change in total chlorine concentration, then the alarm will be initiated.

In some non-limiting embodiments or aspects, the later measured average rate of change in total chlorine concentration after adding both ammonia and chlorine at a weight ratio of chlorine to ammonia of 5:1 or less is determined after a time-delay. The time-delay defines a period of time for which the condition of low rate of change of chlorine residual needs to be maintained before considering that the process operation is abnormal.

As indicated, an alarm will be initiated if the average rate of change in total chlorine concentration is below the second set rate of change in total chlorine concentration and optionally after the previously described time-delay. The alarm can include various methods of alerting a person that there is an abnormal operation of chloramine residual control. In some non-limiting embodiments or aspects, the alarm is a visual and/or audio indication throughout the facility and/or through a computing device including a mobile device such as a cellular phone.

In some preferred and non-limiting embodiments or aspects, the method step of controlling the addition of chlorine and ammonia into the body of water 12 and initiating an alarm is controlled by the following algorithm:

$${\frac{dTCL}{dt}}_{C} < {\frac{dTCL}{dt}}_{min} and elapsed > TD = initiate alarm ,$$

wherein

$${\frac{dTCL}{dt}}_{C}$$

is the observed average rate of change in total chlorine concentration,

$${\frac{dTCL}{dt}}_{min}$$

is the second set rate of change in total chlorine concentration, elapsed is the time elapsed before determining the average rate of change in total chlorine concentration that is compared to the second set rate of change in total chlorine concentration, and TD is the time delay for abnormal detection.

It is appreciated that after fixing any issues that caused the alarm to initiate, chlorine and ammonia can again be added to the body of water 12 until a subsequently obtained water sample is determined to be at or above the predetermined chloramine concentration target level, at which point the programming instructions will cause the controller 40 to stop the supply of chlorine and the supply of ammonia into the body of water 12.

In some preferred and non-limiting embodiments or aspects, the previously described method step of stopping the addition of chlorine and ammonia into the body of water 12 is controlled by the following algorithm in which the predetermined chloramine concentration target level is a residual chloramine concentration set-point based on a single value: z≥x=stop the supply of chlorine and ammonia, where “z” is the residual chloramine concentration determined in a subsequent water sample as chlorine and ammonia are being supplied to the body of water 12, and “x” is a residual chloramine concentration set-point. Thus, the programming instructions can include an algorithm that, when satisfied, will cause the controller 40 to stop automatically engaging (or controlling) a supply of chlorine and a supply of ammonia, and therefore, stop adding chlorine and ammonia to the body of water 12.

In some preferred and non-limiting embodiments or aspects, after fixing any issues that caused the alarm to initiate and adding chlorine and ammonia again to the body of water 12, the controller 40 is programmed to stop the supply of chlorine and ammonia into the body of water 12 when the residual chloramine concentration is above a particular percentage of the residual chloramine concentration set-point. For example, the controller 40 can be programmed to stop the supply of chlorine and ammonia into the body of water 12 when the residual chloramine concentration in a water sample is a percentage selected within a range of 101% to 110% of the residual chloramine concentration set-point, or a percentage selected within a range of 101% to 105% of the residual chloramine concentration set-point.

In such preferred and non-limiting embodiments or aspects, different programming algorithms are used to control when the supply of chlorine and ammonia into the body of water 12 are stopped. For instance, the method step of stopping the supply of chlorine can be controlled by the following algorithm: z>[(t)(x)]=stop the supply of chlorine and ammonia, where “z” is the residual chloramine concentration determined in a subsequent water sample as chlorine and ammonia are being supplied to the body of water 12, “t” is a percentage selected within a range of 101% to 110%, and “x” is the residual chloramine concentration set-point. Thus, the programming instructions can include, or can be modified to include, the above algorithm that, when satisfied, will cause the controller 40 to stop automatically engaging (or controlling) a supply of chlorine and a supply of ammonia, and therefore, stop adding chlorine and ammonia to the body of water 12.

In some preferred and non-limiting embodiments or aspects, the predetermined chloramine concentration target level for controlling and stopping the supply of chlorine and ammonia can include a minimum predetermined chloramine concentration set-point and a maximum predetermined chloramine concentration set-point. The programming instructions will cause the controller 40 to perform certain functions when the chloramine concentration is at or above the minimum predetermined chloramine concentration set-point but below the maximum predetermined chloramine concentration set-point, and to perform different functions when the chloramine concentration is at or above the maximum predetermined chloramine concentration set-point. For instance, the predetermined chloramine concentration target level for controlling and stopping the supply of chlorine and ammonia can comprise: (i) a minimum predetermined chloramine concentration set-point that causes the controller 40 to decrease the feed rate of the chlorine and/or the ammonia into the body of water 12; and (ii) a maximum predetermined chloramine concentration set-point that causes the controller 40 to stop the supply of chlorine and/or the ammonia into the body of water 12.

In some preferred and non-limiting embodiments or aspects, the method step of controlling and stopping the addition of chlorine and ammonia into the body of water 12 is controlled by the following algorithms: z≥x and z≤y=decrease the feed rate of the supply of chlorine and ammonia; and z≥y=stop the supply of chlorine and ammonia, where “z” is the residual chloramine concentration determined in a subsequent water sample as chlorine and ammonia are being supplied to the body of water 12, “x” is a minimum predetermined chloramine concentration set-point, and “y” is a maximum predetermined chloramine concentration set-point. Thus, the programming instructions can include the above algorithms that, when satisfied, will cause the controller 40 to control and/or stop automatically engaging (or controlling) a supply of chlorine and a supply of ammonia, and therefore, modify and/or stop adding chlorine and ammonia to the body of water 12.

As previously mentioned, the predetermined chloramine concentration target level for controlling and stopping the supply of chlorine and ammonia can be based on a total chlorine concentration target level. Thus, it is appreciated that the predetermined chloramine concentration target level used in the previously described method steps and algorithms for controlling and stopping the supply of chlorine and ammonia can be based on a predetermined total chlorine concentration set-point, a percentage of the predetermined total chlorine concentration set-point, or a minimum predetermined total chlorine concentration set-point and a maximum predetermined total chlorine concentration set-point. In such embodiments, the residual chloramine concentration is based on and/or determined from the residual total chlorine concentration in the water samples.

As indicated, the supply of chlorine can be engaged and added to the body of water 12 prior to determining the rate of change in total chlorine. For instance, in some preferred and non-limiting embodiments or aspects, chlorine, and optionally, ammonia are supplied to the body of water 12 after determining that the residual chloramine concentration in the water sample is below the predetermined chloramine concentration target level and before determining the average rate of change in total chlorine concentration. In some preferred and non-limiting embodiments or aspects, the previously described method step of adding chlorine into the body of water 12 before determining the rate of change in total chlorine concentration is controlled by the following algorithm: z<x=add chlorine, where “z” is the residual chloramine concentration determined in the water sample and “x” is the residual chloramine concentration set-point. Thus, the programming instructions can include the above algorithm that, when satisfied, will cause the controller 40 to automatically engage (or control) a supply of chlorine to add chlorine to the body of water 12.

As previously described, the chlorine and ammonia can both be supplied to the body of water 12 after determining that the residual chloramine concentration in the water sample is below the predetermined chloramine concentration target level and before determining the average rate of change in total chlorine concentration. In such embodiments, the chlorine and ammonia are supplied to the body of water 12 at a weight ratio of chlorine to ammonia of greater than 5:1. In some preferred and non-limiting embodiments or aspects, the previously described method step of adding chlorine and ammonia into the body of water 12 before determining the rate of change in total chlorine concentration is controlled by the following algorithm: z<x=add chlorine and ammonia at a weight ratio of chlorine to ammonia of greater than 5:1, where “z” is the residual chloramine concentration determined in the water sample and “x” is the residual chloramine concentration set-point. Thus, the programming instructions can include the above algorithm that, when satisfied, will cause the controller 40 to automatically engage (or control) a supply of chlorine and ammonia to add chlorine and ammonia to the body of water 12 at a weight ratio of chlorine to ammonia of greater than 5:1.

Further, in such embodiments where chlorine and ammonia are supplied to the body of water 12 at a weight ratio of chlorine to ammonia of greater than 5:1, the weight ratio of chlorine to ammonia is adjusted to 5:1 or less when the set rate of change in total chlorine concentration is not satisfied as previously described. Further, the feed rate of ammonia and chlorine supplied at a weight ratio of chlorine to ammonia of greater than 5:1 can be the same or different than the feed rate of ammonia and chlorine supplied at a weight ratio of chlorine to ammonia of greater than 5:1. For example, the feed rate of ammonia and chlorine supplied at a weight ratio of chlorine to ammonia of greater than 5:1 can be the greater or lower than the feed rate of ammonia and chlorine supplied at a weight ratio of chlorine to ammonia of greater than 5:1.

In certain preferred and non-limiting embodiments or aspects, the method uses a chloramine concentration percentage to determine when to engage (or control) and add a supply of chlorine, and optionally, ammonia to the body of water 12 before determining the average rate of change in total chlorine concentration. For instance, the programming instructions can cause the controller 40 to engage (or control) a supply of chlorine, and optionally, ammonia and add the chlorine, and optionally, ammonia to the body of water 12 when it is determined that the residual chloramine concentration in a water sample is below a percentage selected within a range of 99% to 80% of the residual chloramine concentration set-point, or below a percentage selected within a range of 99% to 85% of the residual chloramine concentration set-point, or below a percentage selected within a range of 99% to 90% of the residual chloramine concentration set-point, or below a percentage selected within a range of 99% to 95% of the residual chloramine concentration set-point.

In some preferred and non-limiting embodiments or aspects, the previously described method step of adding chlorine into the body of water 12 based on a chloramine concentration percentage is controlled by the following algorithm: y<[(a)(x)]=add chlorine, where “y” is the residual chloramine concentration determined in the first water sample, “a” is a percentage selected within a range of 99% to 80%, and “x” is the residual chloramine concentration set-point. Further, the previously described method step of adding chlorine and ammonia into the body of water 12 based on a chloramine concentration percentage is controlled by the following algorithm: y<[(a)(x)]=add chlorine and ammonia at a weight ratio of chlorine to ammonia of greater than 5:1, where “y” is the residual chloramine concentration determined in the first water sample, “a” is a percentage selected within a range of 99% to 80%, and “x” is the residual chloramine concentration set-point. Thus, the programming instructions can include, or can be modified to include, the above algorithm that, when satisfied, will cause the controller 40 to automatically engage (or control) a supply of chlorine to add chlorine to the body of water 12.

It is appreciated that the predetermined chloramine concentration target level used prior to determining the rate of change in total chlorine concentration can be based on a total chlorine concentration target level. As such, the predetermined chloramine concentration target level used in the previously described method steps and algorithms and which is used before determining the rate of change in total chlorine concentration can be based on a predetermined total chlorine concentration set-point or a percentage of the predetermined total chlorine concentration set-point. In such embodiments, the residual chloramine concentration is based on and/or determined from the residual total chlorine concentration in the water samples.

In some preferred and non-limiting embodiments or aspects, chlorine and ammonia are not added before determining the rate of change in total chlorine concentration. In such embodiments, both chlorine and ammonia can be directly added after determining that the desired set rate of change in total chlorine concentration is not achieved.

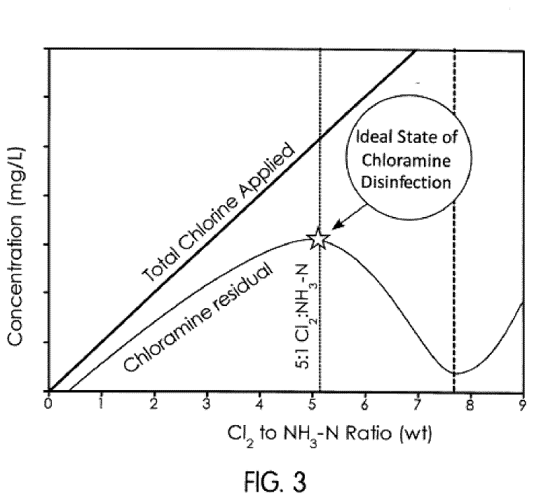

The method of the present invention works in accordance with the chloramine breakpoint curve, shown in FIG. 3. In particular, the previously described steps are used to achieve and maintain an ideal state of monochloramine disinfectant by predicting where the chloramine concentration in the body of water 12 resides along the breakpoint curve, the rate at which chloramine concentration is increasing and decreasing in the body of water 12 over time, and adjusting the input of chlorine or chlorine and ammonia into the body of water 12 to achieve and maintain a position at or near the ideal state. As shown in FIG. 3, the ideal state (i.e., the maximum monochloramine concentration obtainable in a body of water 12) is typically achieved at a weight ratio of chlorine (Cl₂) to ammonia-nitrogen (NH₃—N) of 5:1.

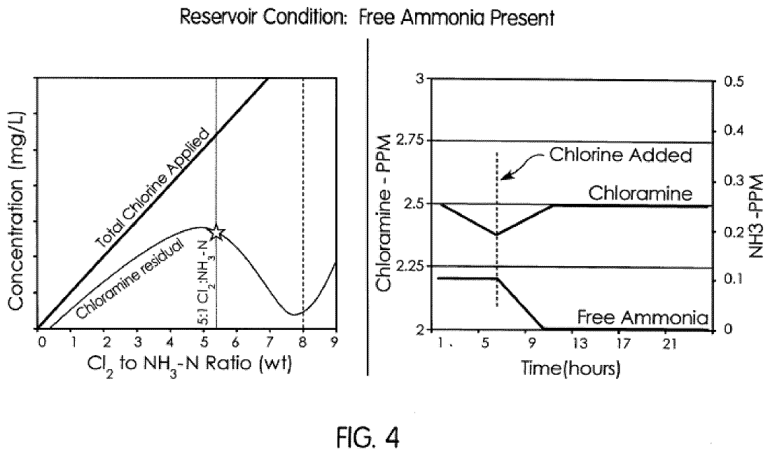

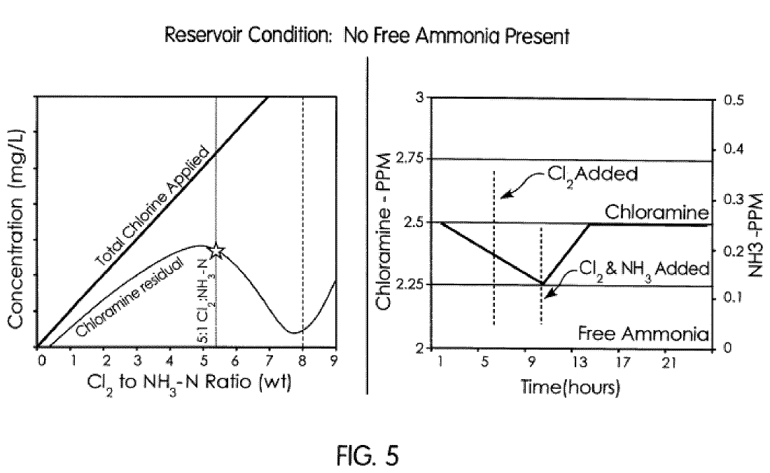

Referring to FIGS. 4 and 5, and in one preferred and non-limiting embodiment or aspect, the method includes at least two modes, or stages, in view of the chloramine breakpoint curve. In the first mode shown in FIG. 4, it is assumed that free ammonia is present in the body of water 12. During the first mode, water samples are periodically drawn from the body of water 12 and analyzed to determine the chloramine concentration. In one preferred and non-limiting embodiment or aspect, this determination is accomplished by measuring the total chlorine present in the sample using a total chlorine analyzer, such as the total chlorine analyzer commercially available from ProMinent Fluid Controls, Inc. of Pittsburgh, PA. If the system 10 determines that the total chlorine levels measured are in decline, the controller 40 can be configured to engage (or control) the treatment tubes 22 or 24 to add chlorine, and optionally ammonia at a weight ratio of chlorine to ammonia of greater than 5:1, to the body of water 12. Newly added chlorine will react with the free ammonia to generate chloramine, thus increasing the concentration of chloramine in the body of water 12 and reducing the concentration of free ammonia, as reflected in FIG. 4. Once the residual chloramine concentration target level is reestablished, or established in the first instance, the addition of chlorine, and optionally ammonia when also supplied, can cease.

In the second mode or stage of the control method as shown in FIG. 5, no free ammonia is present in the body of water 12. Because no free ammonia is present, the addition of chlorine, and optionally ammonia at a weight ratio of chlorine to ammonia of greater than 5:1, in response to a recognized drop in the chloramine concentration will result not in an upswing (or increase) in the chloramine concentration, as in the first mode described above, but rather in a further reduction in the chloramine concentration. This is caused by the absence of a sufficient amount of free ammonia in the body of water 12, which precludes the formation of chloramine through a reaction between the added chlorine and free ammonia. If, after the addition of chlorine, and optionally ammonia at a weight ratio of chlorine to ammonia of greater than 5:1, in the first mode, the chloramine concentration does not increase after a sufficient amount of time and the rate of change in the total chloramine concentration is below the set rate of change in total chlorine concentration, the system 10 can conclude that a sufficient amount of free ammonia is absent from the body of water 12. In response, the controller 40 is configured to engage (or control) a source of chlorine and a source of ammonia to inject into the body of water 12 at a weight ratio of chlorine to ammonia of 5:1 or less, as reflected in FIG. 5. The ammonia and chlorine at a weight ratio of chlorine to ammonia of 5:1 or less can continue being added until analysis of water samples extracted from the body of water 12 determines that the residual chloramine concentration target level has been reestablished (or established). In some preferred and non-limiting embodiments or aspects, ammonia and chlorine are added at a weight ratio of chlorine to ammonia of 5:1 or less for a period a time and then stopped to allow for a low concentration of free ammonia without achieving the residual chloramine concentration target level.

It is appreciated that the second mode is initiated by the ability of the system 10 to predict the location of the chloramine reaction on the break point curve and the rate at which the chloramine concentration is increasing or decreasing. For instance, if the measured total chlorine residual concentration continues to decrease as chlorine, and optionally ammonia at a weight ratio of chlorine to ammonia of greater than 5:1, is added, the system 10 can conclude that a sufficient amount of free ammonia is not present and that the residual chloramine concentration is decreasing past the ideal state shown in FIG. 3. As a result, the second mode is initiated and the controller 40 will add chlorine and ammonia into the body of water 12 at a weight ratio of chlorine to ammonia of 5:1 or less.

In some preferred and non-limiting embodiments or aspects, the second mode or stage of the control method is determined without engaging the supply of chlorine, or chlorine and ammonia at a weight ratio of chlorine to ammonia of greater than 5:1, when the total chlorine levels measured are in decline and the rate of change in the total concentration is below the first set rate of change in total chlorine concentration. In response, the controller 40 is configured to engage (or control) a source of chlorine and a source of ammonia to inject into the body of water 12 at a weight ratio of chlorine to ammonia of 5:1 or less. If the rate of change in the total concentration is below the second set rate of change in total chlorine concentration in this second mode or stage, then the alarm will be initiated for abnormal operation.

It is appreciated that the rate of change in total chlorine concentration can be used to determine the chloramine concentration such as the concentration of monochloramine, di-chloramine, or the like. For instance, and as previously explained, the rate of change in total chlorine concentration can be used to determine if the chemicals in the body of water are in a state of monochloramine or di-chloramine, which can then be used to determine chlorine and ammonia feed rate and/or a ratio of chlorine to ammonia that should be supplied to the body of water 12.

As indicated, any of the previously described method steps, or combination of steps, can be used to establish, reestablish, and maintain a desired residual chloramine level within the body of water 12. In one preferred and non-limiting embodiment or aspect, at least one of the previously described method steps, or combination of steps, are used to establish or reestablish a predetermined chloramine concentration target level. After the desired predetermined chloramine concentration target level is established or reestablished to complete a first treatment cycle, a different algorithm can be used to reestablish the desired residual chloramine concentration in subsequent treatment cycles.

The feed rate of chlorine and/or ammonia in any of the previously described steps can be determined from the reservoir 14 water volume and dwell time. As used herein, “dwell time” refers to the rate at which water volume changes in the reservoir 14. The feed rate of the chlorine and ammonia can also be controlled by the speed at which the metering pumps distribute the chlorine and ammonia into the body of water 12. For example, the metering pumps can distribute chlorine and ammonia at a maximum speed rate. The metering pumps can also be reduced to half (i.e., 50%) of the maximum speed rate to adjust the feed rate of chlorine and ammonia.

The method of automatically controlling chloramine concentration described herein allows for a desired amount of chloramine in a body of water 12 to be effectively maintained without directly measuring or initially adding free ammonia. Further, by monitoring the rate of change in the total concentration in view of set rates of change in total chlorine concentration as previously described, abnormal operation of chloramine residual control can be quickly determined and which are extremely difficult to rapidly detect with other fail-safes. For example, a sudden leak from the chlorine line just before tank penetration while dosing chlorine and ammonia normally can occur, while various sub-systems such as the chemical dosing pumps, discharge pressure, mixing system and water quality may work as expected. As a result, the process goal will not be achieved because of the chlorine leak, ammonia will likely be fed alone for a long period of time until the dosing is stopped such as by the operator. By using the methods and systems described herein that trigger an alarm based on monitoring the rate of change in the total concentration in view of set rates of change in total chlorine concentration, the previously described abnormal operation of chloramine residual control can be quickly and easily determined.

Whereas particular embodiments of this invention have been described above for purposes of illustration, it will be evident to those skilled in the art that numerous variations of the details of the present invention may be made without departing from the invention as defined in the appended claims.

The invention claimed is:

1. A method of automatically detecting abnormal operation of chloramine residual control in a body of water contained in a reservoir, the method comprising:
a) determining residual chloramine concentration in a water sample obtained from the body of water;
b) determining an average rate of change in total chlorine concentration when the residual chloramine concentration is below a predetermined target chloramine concentration level;
c) automatically engaging a supply of ammonia and a supply of chlorine to add both ammonia and chlorine to the body of water at a weight ratio of chlorine to ammonia of 5:1 or less when the average rate of change in total chlorine concentration is below a first set rate of change in total chlorine concentration; and
d) initiating an alarm to indicate an abnormal operation of residual chloramine residual in the body of water if, after step c), an average rate of change in total chlorine concentration is below a second set rate of change in total chlorine concentration.

2. The method of claim 1, wherein the first set rate of change in total chlorine concentration is equal to or greater than the second set rate of change in total chlorine concentration.

3. The method of claim 1, wherein the average rate of change in total chlorine concentration is determined by measuring the change in residual total chlorine concentration in consecutively obtained water samples over a fixed period of time.

4. The method of claim 1, wherein the residual chloramine concentration is based on a residual total chlorine concentration.

5. The method of claim 1, wherein the average rate of change in total chlorine concentration in step d) is determined after a time-delay following step c).

6. The method of claim 5, wherein the alarm is initiated in step d) when the following algorithm is satisfied:
$${\frac{dTCL}{dt}}_{C} < {\frac{dTCL}{dt}}_{min} and elapsed > TD = initiate alarm ,$$
wherein
$${\frac{dTCL}{dt}}_{C}$$
is the observed average rate of change in total chlorine concentration,
$${\frac{dTCL}{dt}}_{min}$$
is the second set rate of change in total chlorine concentration, elapsed is the time elapsed before determining the average rate of change in total chlorine concentration that is compared to the second set rate of change in total chlorine concentration, and TD is the time delay for abnormal detection.

7. The method of claim 1, further comprising automatically engaging a supply of chlorine only to add only chlorine to the body of water, or engaging a supply of ammonia and a supply of chlorine to add both ammonia and chlorine to the body of water at a weight ratio of chlorine to ammonia of greater than 5:1, prior to step b) if the residual chloramine concentration in the water sample obtained from the body of water in step a) is below the predetermined target chloramine concentration level.

8. A treatment delivery and control system for detecting abnormal operation of chloramine residual control in a body of water contained in a reservoir comprising:
a chemical dosing assembly;
a water sampling assembly configured to extract a water sample from the body of water at different points in time;
one or more analyzers in fluid communication with the water sampling assembly and configured to determine at least total chlorine concentration in the water samples;
a controller in operable communication with the one or more analyzers; and
one or more computer-readable storage mediums in operable communication with the controller and containing programming instructions that, when executed, cause the controller to:
a) determine residual chloramine concentration in a water sample obtained from the body of water;
b) determine an average rate of change in total chlorine concentration when the residual chloramine concentration is below a predetermined target chloramine concentration level;
c) automatically engage a supply of ammonia and a supply of chlorine to add both ammonia and chlorine to the body of water at a weight ratio of chlorine to ammonia of 5:1 or less when an average rate of change in total chlorine concentration is below a first set rate of change in total chlorine concentration; and
d) initiate an alarm to indicate an abnormal operation of chloramine residual in the body of water if, after step c), an average rate of change in total chlorine concentration is below a second set rate of change in total chlorine concentration.

9. The system of claim 8, wherein the chemical dosing assembly is at least partially submerged in the body of water.

10. The system of claim 8, wherein the first set rate of change in total chlorine concentration is equal to or greater than the second set rate of change in total chlorine concentration.

11. The system of claim 8, wherein the average rate of change in total chlorine concentration is determined by measuring the change in residual total chlorine concentration in consecutively obtained water samples over a fixed period of time.

12. The system of claim 8, wherein the residual chloramine concentration is based on a residual total chlorine concentration.

13. The system of claim 8, wherein the average rate of change in total chlorine concentration in step d) is determined after a time-delay following step c).

14. The system of claim 13, wherein the alarm is initiated in step d) when the following algorithm is satisfied:
$${\frac{dTCL}{dt}}_{C} < {\frac{dTCL}{dt}}_{min} and elapsed > TD = initiate alarm ,$$
wherein
$${\frac{dTCL}{dt}}_{C}$$
is the observed average rate of change in total chlorine concentration,
$${\frac{dTCL}{dt}}_{min}$$
is the second set rate of change in total chlorine concentration, elapsed is the time elapsed before determining the average rate of change in total chlorine concentration that is compared to the second set rate of change in total chlorine concentration, and TD is the time delay for abnormal detection.

15. The system of claim 8, wherein the one or more computer-readable storage mediums in operable communication with the controller further contains programming instructions that, when executed, cause the controller to automatically engage a supply of chlorine only to add only chlorine to the body of water, or engage a supply of ammonia and a supply of chlorine to add both ammonia and chlorine to the body of water at a weight ratio of chlorine to ammonia of greater than 5:1, prior to step b) if the residual chloramine concentration in the water sample obtained from the body of water in step a) is below the predetermined target chloramine concentration level.
